(19) United States
(12) Patent Application Publication
(10) Pub. No.: US 2022/0387285 A1
(43) Pub. Date: Dec. 8, 2022

(54) HAIR CARE FORMULATION

(71) Applicant: KDC/ONE SWALLOWFIELD LIMITED, Wellington (GB)

(72) Inventors: Daniel Pickering, Wellington (GB); Conan Fisher, Wellinton (GB)

(73) Assignee: KDC/ONE SWALLOWFIELD LIMITED, Wellington (GB)

(21) Appl. No.: 17/770,038

(22) PCT Filed: Oct. 19, 2020
(86) PCT No.: PCT/GB2020/052627

§ 371 Date: Apr. 18, 2022

(30) Foreign Application Priority Data
Oct. 19, 2019 (GB) 1915166.1

(51) Int. Cl. A61K 8/73; A61K 31/045; A61K 8/04; A61K 31/205; A61Q 5/00; A61Q 5/02

(52) U.S. Cl. CPC A61K 8/732; A61K 31/045; A61K 8/046; A61K 31/205; A61Q 5/008; A61Q 5/02

(57) ABSTRACT

A non-rinse hair care formulation is provided and comprises an oil adsorber and water.

The present invention relates generally to hair care formulations and particularly, although not exclusively, to shampoos, conditioners and the like.

Dry shampoo is a type of hair product that can reduce oil, grease, and dirt in hair. Unlike wet shampoos and conditioners, dry shampoo can be applied to hair whilst it is dry—hence the name.

Dry shampoo can be administered as a powder, where all the ingredients of dry shampoo are combined together and applied to the scalp with the hand, or through the aerosol form where the dry shampoo is sprayed directly onto the head. In the aerosol form, the powders comprising the dry shampoo are dispersed throughout liquified gas inside a can; when the button is depressed, opening the valve, the liquified gas and powders inside are released, forming the aerosol that lands on the hair and scalp.

Dry shampoo sprays are conventionally an anhydrous/dry powder spray i.e. powdered starch or similar, suspended in liquified propellant, completely or almost completely in the absence of water, and sprayed onto the hair to absorb excess oils and refresh hair between washes. These sprays are by their nature are mattifying and whitening, leaving a powdering residue, and making it impossible to achieve any sort of ‘shine’ claim.

There are a number of disadvantages of this formulation type, including:

Whitening effect

Mattifying effect

Powdery spray

Not possible to add water soluble actives

High VOC content

An aspect of the present invention provides a non-rinse hair care formulation comprising an oil adsorber and water.

An aspect of the present invention provides a non-rinse hair care formulation comprising an oil absorber and water.

An aspect provided a hair care formulation comprising an oil adsorber and water.

The adsorber may be a natural oil, e.g. sebum, adsorber.

An aspect provides water-based dry shampoo.

An aspect of the present invention provides a non-rinse hair care formulation comprising starch and water.

Water-based formulations formed in accordance with the present invention can seek to addresses the disadvantages of known such hair care products.

Although using water in ‘dry shampoo’ appears counter-intuitive, the water allows the starch to be delivered to the hair shaft within a droplet, first coating the hair, then quickly drying to leave a very fine and even coating of starch compared to dry powder particles are sprayed on the hair.

In some aspects and embodiments the concept formulation is to deliver adsorbent (e.g. an oil adsorbing ingredient/powder, such as starch) in a very fine water mist spray. This way, the starch particles are finer and partially or wholly dissolved on the water base, so when the product is sprayed it quickly dries to leave an ‘invisible’ coating of starch. It then adsorbs/absorbs/soaks up oils in the same way as a conventional dry shampoo, but without the whitening/mattifying effect and we can even add a water soluble ingredient to give an ‘added shine’ claim.

Adsorbent (e.g. starch) may be present in the range 1% to 25% by mass, for example starch may be present in the range 3% to 12%, or may be present in the range 10% to 15% by mass.

The formulation may further comprise ethanol. For example, up to 30% of the water content may be replaced by ethanol. In some embodiments ethanol is present in the range 15% to 20% by mass. Ethanol can be used to improve drying time and/or affect droplet size, for example by adding some ethanol to the water dispersion of starch, the droplet size can be reduced further, reducing drying time. Ethanol can also be used to protect the formula from microbial growth.

In some aspects and embodiments formulation may be dispensed/dispersed using a pump spray and/or a propellant. The propellant could be compressed air, thus keeping the VOC content to a minimum. In some embodiments VOC content is restricted to no more than 50%.

In some embodiments the propellant is one or more of propane, n-butane, isobutane, dimethyl ether and methyl ethyl ether.

In some embodiments a water soluble liquified propellant such as dimethylether may be used. The propellant may be present at about 50% by mass, giving an even finer mist and even faster drying time, adding to improved consumer acceptance.

Compositions may further comprise betaine as a shine ingredient. Betaine may be present in the range 0% to 5% by mass, for example 0.5% to 2% by mass.

Compositions may further comprise one or more hair conditioner ingredients.

Hair conditioner ingredients may comprise one or more of: acidifiers, fragrance, acidity regulators, antistatic agents, detanglers, glossers, light-reflecting chemicals, lubricants, moisturisers, oils, preservatives, reconstructors, sequestrants, sunscreen, thermal protectors and solubiliser.

Regulators can be used to maintain the conditioner's pH, for example at about 3.5. In contact with acidic environment, the hair's somewhat scaly surface tightens up, as the hydrogen bonds between the keratin molecules are strengthened. Antistatic agents may be used. Detanglers, may be used to modify the hair surface pH as acidifiers, or by coating it with polymers, as glossers. Glossers may be used. Light-reflecting chemicals which bind to the hair surface may be used. Styling polymers, such as PVP, VP/VA Copolymer or amphomer resin, may be used. Lubricants, such as fatty alcohols, panthenol, dimethicone, etc may be used. Moisturisers may be used to hold moisture in the hair (usually these contain high proportions of humectants); these could also be provided by natural oils such as almond oil. Oils (essential fatty acids) may be used, which can help dry/porous hair become softer and more pliable. The scalp produces a natural oil called sebum. EFAs are the closest thing to natural sebum (sebum contains EFAs). Preservatives may be used. Reconstructors, usually containing hydrolysed protein may be used. Sequestrants, for better function in hard water, may be used. Sunscreen, for protection against protein degradation and colour loss, may be used, for example benzophenone-4, ethylhexyl methoxycinnamate, or cinnamidopyltrimonium chloride. Surfactants may be used. Hair conditioners often contain cationic surfactants, which don't wash out completely, because their hydrophilic ends strongly bind to keratin. Thermal protectors may be used; they are usually heat-absorbing polymers, shielding the hair against excessive heat, caused by, e.g., blow-drying, curling irons or hot rollers

Hair conditioning ingredient/s may, for example, be present at up to 1%, 2%, 3% or 5% by mass.

The starch may be modified or unmodified.

The starch may comprise one or more of corn starch, modified corn starch, tapioca starch, rice starch or modified rice starch.

In one embodiment Corn PO4 PH″B, manufactured by Agrana (INCI name Distarch phosphate) may be used.

A further aspect provides a dry shampoo formulation comprising a formulation as described herein.

Dry shampoo is a type of hair product that can reduce oil, grease, and dirt in hair. Unlike wet shampoos and conditioners, dry shampoo can be applied to the hair while it is dry—hence the name. Dry shampoo doesn't need to be washed out of the hair, and it's typically applied to the crown of the head and other areas where oil and shine may visibly collect.

Also provided is a hair care pack comprising a formulation as described herein.

The pack may comprise an aerosol container (such as a can).

The pack may comprise a propellant, such as compressed air and/or liquified propellant.

Formulations may be provided as a starch spray and delivered by liquefied propellant (such as dimethylether) or compressed air as propellant.

Different starch sources and chemically modified starches may be used Small additions of solvents may be used.

A water soluble liquified propellant may be used to improve drying time and a ‘drier’ spray feel.

Percentages and other expressions of amounts of ingredients may be by mass and/or by volume. In some embodiments all the percentages/ingredient levels are by mass.

Different aspects and embodiments can be used together or separately.

Embodiments of the present invention are more particularly described, by way of non-limiting example, below.

The example embodiments are shown in sufficient detail to enable those of ordinary skill in the art to embody and implement the systems and processes herein described. It is important to understand that embodiments can be provided in many alternate forms and should not be construed as limited to the examples set forth herein.

Embodiments can be modified in various ways and take on various alternative forms. There is no intent to limit to the particular forms disclosed. On the contrary, all modifications, equivalents, and alternatives falling within the scope of the appended claims should be included.

Unless otherwise defined, all terms (including technical and scientific terms) used herein are to be interpreted as is customary in the art.

It will be further understood that terms in common usage should also be interpreted as is customary in the relevant art and not in an idealised or overly formal sense unless expressly so defined herein.

One of ordinary skill in the art will appreciate the many possible applications and variations of the present invention based on the following examples of possible embodiments of the present invention.

Some aspects and embodiments may be based on the following.

VOC Level

In some embodiments the VOC level for the formulation is no more than 50%.

Water

Water

Filtered water

Deionised water

Up to 95%

50-95%

20-95%

Starch or Other Adsorbent Powder

Example Range: 1-25%, preferably 10-15%. For example 15%.

Example Range: 25%, preferably 3-12%. For example 10%.

Starched can be unmodified or chemically modified:

Corn starch

Modified corn starch (Distarch phosphate)

Tapioca starch

Modified tapioca starch

Rice starch

Modified rice starch

Other adsorbent powder could be zeolite or insoluble polyvinylpolypyrrolidone (PVP).

In some embodiments starch is used. In some embodiment other oil adsorbing powders are alternatively or additionally used—such as zeolite or insoluble PVP.

Starch Example: Corn PO4 PH″B, manufactured by Agrana. INCI name Distarch phosphate.

Examples of other materials: Advantage Revive, manufactured by Ashland (INCI name PVP);

Sylosiv A4, manufactured by Grace GmbH (INCI name Zeolite).

Ethanol

Example Range: 0-30%, preferably 15-20%

Example Range: 0-50%, preferably 0-12%

Ethanol may be removed from some core LPG formulations. In some embodiments a drier spray can be achieved by replacing ethanol with more DME propellant (whilst keeping the total VOC content generally the same). Ethanol may be used in compressed air formulations.

Propellant

Compressed air: pressurised sufficiently to give good break-up in spray, to the end of pack. Product is added, then headspace is pressurised.

Dimethylether: Example Range: 10-55%, preferably 35-45%
Example Range: 10-55%, preferably 40-50%

Liquid propellant may become part of the formulation.

Suspending Agent

Fumed silica (trade name Wacker HDK N20, manufactured by Wacker Silicones) Example Range: 0-1%, preferably 0.2-0.5%

Suspending agent (e.g. silica) may, for example, be included to improve stability and spray consistency.

Shine Ingredient

Natural betaine (Trade name Betefin)

Range: 0-5%, preferably 0.5-2%.

Conditioning

Water soluble, cationic hair conditioning ingredients such as cetrimonium chloride, can be added to the formulation to give enhanced conditioning, anti-frizz claims. Up to 1%.

Pigments and/or Colourants and/or Dyes

Solid pigments, e.g. iron oxides CI 77491, CI 77499, CI 77492.

Soluble dyes e.g. CI 42090, CI 14700, CI 47005.

Drying Time

In some embodiments the drying time upon application is: 0-15 seconds; 15-30 seconds; 30-60 seconds; a maximum of 60 seconds. The term “drying time” may mean the average time a user deems has passed before their hair is “dry”.

Concentrate and Propellant to Concentrate Ratios for Liquified Product

| | | | | | DME:Conc |
|---|---|---|---|---|---|
| Ingredient | Water | Starch | Betafin | Ethanol | ratio |
| Minimum levels for | Up to 100% | 10% in Conc* | 1% in Conc* | 0% in Conc* | 54.5:45.5 |
| functional product | | | | | |
| Ideal level for good | Up to 100% | 15% in Conc* | 2% in Conc* | 20% in Conc* | 43:57 |
| product performance | | | | | |
| Max levels for a | Up to 100% | 25% in Conc* | 5% in Conc* | 30% in Conc* | 35:65 |
| positive use product | | | | | |

Propellant Ratios for Liquified Product

| Ingredient | Water | Starch | Betafin | Ethanol | DME |
|---|---|---|---|---|---|
| Minimum levels for | Up to 100% | 5% | 1% | 0% | 35% |
| functional product | | | | | |
| Ideal level for good | Up to 100% | 10% | 2% | 0% | 50% |
| product performance | | | | | |
| Max levels for a | Up to 100% | 15% | 5% | 15% | 55% |
| positive use product | | | | | |

DME/Concentrate levels determined to produce a product with an overall VOC level of about in the range 45-55%, for example about 50%, or about 54.5%.

Example Core LPG Formula

| Ingredient | Level in finished goods |
|---|---|
| DEIONISED WATER | 35.055% |
| CORN PO4 PH“B” | 8.55% |
| (Distarch phosphate) | |
| BETAFIN BP20 | 1.14% |
| (Betaine Anhydrous) | |
| ETHANOL B 750 P | 11.4% |
| (DEB100) | |
| Other (fragrance, | 0.855% |
| extract, solubiliser) | |
| DME | 43% |
| (DIMETHYL ETHER) | |

Example Core LPG Formula

| Ingredient | Level in finished goods |
|---|---|
| DEIONISED WATER | To 100% |
| CORN PO4 PH“B” | 10% |
| (Distarch phosphate) | |
| BETAFIN BP20 | 2% |
| (Betaine Anhydrous) | |
| Wacker HDK N20 | 0.25% |
| (silica) | |
| Other (fragrance, | 1.25% |
| extract, solubiliser) | |
| DME | 50% |
| (DIMETHYL ETHER) | |

Example Formulation for Compressed Gas Product

| Ingredient | Water | Starch | Betafin | Ethanol |
|---|---|---|---|---|
| Minimum levels for | Up to 100% | 10% | 1% | 0% |
| functional product | | | | |
| Ideal level for goo | Up to 100% | 15% | 2% | 30% |
| product performance | | | | |
| Max levels for a | Up to 100% | 25% | 5% | 50% |
| positive use product | | | | |

Higher ethanol Levels to allow for loss of volatility from having no LPG in a can.

Example Core Compressed Gas Formula

| Ingredient | Level in finished goods |
|---|---|
| DEIONISED WATER | 51.5% |
| CORN PO4 PH“B” | 15% |
| (Distarch phosphate) | |
| BETAFIN BP20 | 2% |
| (Betaine Anhydrous) | |
| ETHANOL B 750 P | 30% |
| (DEB100) | |
| Other (fragrance, | 1.5% |
| extract, solubiliser) | |

Percentages and other expressions of amounts of ingredients may be by mass and/or by volume. In some embodiments all the percentages/ingredient levels are by mass.

Levels may be overall, finished goods levels.

Example Liquified Propellant Version:

| | |
|---|---|
| Dimethyl ether | 50.00% |
| Water | 36.50% |
| Distarch phosphate (maize) | 10.00% |
| Betaine | 2.00% |
| Polyglyceryl-4 Caprate | 0.50% |
| Phenoxyethanol | 0.35% |
| Parfum | 0.25% |
| Silica | 0.25% |
| Chlorphenesin | 0.10% |
| Glycerin | 0.05% |

Example Compressed Air Version:

| | |
|---|---|
| Ethanol | 45.00% |
| Water | 41.00% |
| Distarch phosphate (maize) | 10.00% |
| Betaine | 2.00% |
| Phenoxyethanol | 0.70% |
| Polyglyceryl-4 Caprate | 0.50% |
| Parfum | 0.25% |
| Silica | 0.25% |
| Chlorphenesin | 0.20% |
| Glycerin | 0.10% |

The present inventions can be embodied in other specific apparatus and/or methods. The described embodiments are to be considered in all respects as illustrative and not restrictive. In particular, the scope of the invention is indicated by the appended claims rather than by the description and figures herein. All changes that come within the meaning and range of equivalency of the claims are to be embraced within their scope.

1. A water-based, non-rinse hair care spray formulation comprising: an oil adsorber; and water in a range 20-95%.

2. The formulation of claim 1, in which adsorber is present in a range 1% to 25% by mass.

3. The formulation of claim 2, in which adsorber is present in a range 3% to 12% by mass.

4. The formulation of claim 1, in which the adsorber comprises starch.

5. The formulation of claim 1, further comprising ethanol.

6. The formulation of claim 5, in which ethanol is present in a range 0% to 50% by mass or present in a range 0% to 12% by mass.

7. (canceled)

8. The formulation of claim 1, further comprising compressed air as a propellant.

9. The formulation of claim 1, further comprising a gaseous propellant.

10. The formulation of claim 9, in which the propellant is one or more of propane, n-butane, isobutane, dimethyl ether and methyl ethyl ether.

11. The formulation of claim 1, further comprising betaine.

12. The formulation of claim 11, in which betaine is present in a range 0% to 5% by mass or in a range 0.5% to 2% by mass.

13. (canceled)

14. The formulation of claim 1, further comprising one or more hair conditioner ingredients selected from the group comprising: acidifiers, fragrance, acidity regulators, antistatic agents, detanglers, glossers, light-reflecting chemicals, lubricants, moisturisers, oils, preservatives, reconstructors, sequestrants, sunscreen, suspending agents, thermal protectors and solubiliser.

15. (canceled)

16. (canceled)

17. The formulation of claim 1, in which VOC content is restricted to no more than 50%.

18. A dry shampoo formulation comprising the formulation of claim 1.

19. A hair care pack comprising the formulation of claim 1.

20. The pack of claim 19, comprising an aerosol container and configured to deliver the oil adsorber in a water mist spray.

21. The pack of claim 20, comprising a propellant.

22. The pack of claim 21, in which the propellant is compressed air or in which the propellant is dimethyl ether.

23. (canceled)

24. The formulation of claim 4, in which the adsorber comprises one or more of corn starch, modified corn starch, tapioca starch, rice starch and modified rice starch.

25. The formulation of claim 1, in which the adsorber comprises zeolite or insoluble PVP.
